(19) United States
(12) Patent Application Publication
(10) Pub. No.: US 2024/0173249 A1
(43) Pub. Date: May 30, 2024

(54) COSMETIC COMPOSITION CONTAINING BEE POLLEN EXOSOME

(71) Applicants: LG HOUSEHOLD & HEALTH CARE LTD., Seoul (KR); MORECHEM CO., LTD., Seoul (KR)

(72) Inventors: Younjung LEE, Seoul (KR); Taeho JI, Seoul (KR); Na Yoon KIM, Seoul (KR); IL WOO JOO, Yongin-si (KR); Ji Ean LEE, Yongin-si (KR); Da Jeong KIM, Yongin-si (KR)

(73) Assignees: LG HOUSEHOLD & HEALTH CARE LTD., Seoul (KR); MORECHEM CO., LTD., Seoul (KR)

(21) Appl. No.: 18/521,016

(22) Filed: Nov. 28, 2023

(30) Foreign Application Priority Data
Nov. 29, 2022 (KR) 10-2022-0163348
Nov. 15, 2023 (KR) 10-2023-0158409

(51) Int. Cl. A61K 8/98; A61Q 19/00; A61Q 19/02; A61Q 19/08

(52) U.S. Cl. CPC A61K 8/988; A61Q 19/00; A61Q 19/02; A61Q 19/08

(57) ABSTRACT

The present invention relates to a cosmetic composition including a bee pollen exosome, more particularly, a cosmetic composition including the bee pollen exosome as an active ingredient, the cosmetic composition having excellent effects such as skin whitening, skin elasticity enhancement, wrinkle improvement, skin moisturizing, skin regeneration, skin soothing, skin radiance improvement or anti-inflammation, etc. Further, provided are a functional food composition and a quasi-drug composition, each including the bee pollen exosome.

### TECHNICAL FIELD

The present invention relates to a cosmetic composition including a bee pollen exosome, more particularly, a cosmetic composition including the bee pollen exosome as an active ingredient, the cosmetic composition having excellent effects such as skin whitening, skin elasticity enhancement, wrinkle improvement, skin moisturizing, skin regeneration, skin soothing, skin radiance improvement or anti-inflammation, etc. Further, the present invention relates to a functional food composition and a quasi-drug composition, each including the bee pollen exosome.

### BACKGROUND ART

There are many causes of skin aging, and in particular, ultraviolet rays and reactive oxygen species generated through respiration are considered the most important causes of skin aging. Reactive oxygen species lead to damage of skin cells and tissues. Normally, reactive oxygen species create balance by killing abnormal and aged cells. However, when excessively generated, they destroy the skin's antioxidant defense network, cause lipid peroxidation and protein oxidation, destroy interstitial components, and destroy skin proteins such as collagen and elastin, etc., resulting in skin aging such as reduced elasticity, wrinkles, pigmentation, etc. There has been recent research demonstrating a positive correlation between protein intake and an increase in skin moisture, and from a perspective of nutrition, it can be inferred that intake of high-quality proteins is closely related to skin health. However, protein intake through foods has only an insignificant effect on the skin.

More than 500 types of plant extracts with specific pharmacological effects such as antioxidation and anti-aging are known in Korea, but most of them have not been verified for their effects on the skin. Since numerous components exist at various proportions in plant extracts, it is difficult to find an effective substance with certain effects. In addition, an effective substance among plant ingredients may act alone as a single ingredient, but the effect may only appear through the interaction of several ingredients. Moreover, it is known that various existing plant extracts have difficulty in passing through the skin barrier, and thus the effect by simply applying them to the skin is limited.

Accordingly, there is a need for the development of substances capable of maximizing all of skin whitening, anti-oxidation, skin regeneration, skin soothing, skin radiance improvement, anti-inflammatory and anti-wrinkle effects while minimizing side effects. There is also an increasing demand for cosmetic compositions that can be used without restrictions on the content because they have fewer harmful factors such as skin irritation, cytotoxicity, etc.

On the other hand, exosomes are several tens to hundreds of nanometer-sized vesicles consisting of a double phospholipid membrane having the same structure as that of the cell membrane. Exosomes are intercellular signaling mediators secreted by cells, and it is known that various cellular signals transmitted through exosomes regulate cell behaviors including activation, growth, migration, differentiation, dedifferentiation, apoptosis, and necrosis of target cells.

Exosomes contain specific genetic materials and bioactive factors depending on the nature and state of cells from which the exosomes are derived. Exosomes isolated from animal stem cells have safety issues in that the factors contained in the exosomes may exhibit effects other than those intended, and stem cells may be contained during isolation of the exosomes. However, the safety issues of exosomes derived from animal cells can be solved by using exosomes of plant cells. For this reason, studies on exosomes of plant cells have been actively conducted, but is not still entirely satisfactory.

In particular, a pollen extract is known to mainly have antifungal, antibacterial, and antiviral effects. Although bee pollens contain exosome components, bee pollen-derived exosome components have not yet been used as a cosmetic ingredient, and there is little research on their skin anti-aging effect.

### PRIOR ART DOCUMENTS

### Patent Documents

(Patent Document 1) KR 10-2021-0135138 A
(Patent Document 2) KR 10-2022-0104655 A

### DISCLOSURE

### Technical Problem

An object of the present invention is to provide a cosmetic composition including a bee pollen exosome as an active ingredient.

Another object of the present invention is to provide a functional food composition including a bee pollen exosome as an active ingredient.

Still another object of the present invention is to provide a quasi-drug composition including a bee pollen exosome as an active ingredient.

Still another object of the present invention is to provide a method of preparing a bee pollen exosome.

Still another object of the present invention is to provide a bee pollen exosome which is prepared by a method of preparing the bee pollen exosome.

Still another object of the present invention is to provide a composition including a bee pollen exosome.

Still another object of the present invention is to provide a method of improving the skin, the method including the step of applying, to a subject′ skin, a composition including a bee pollen exosome as an active ingredient.

Still another object of the present invention is to provide use of a bee pollen exosome or a composition including the same in improving the skin.

### Technical Solution

The present invention will be described in detail as follows. Meanwhile, each description and embodiment disclosed in this disclosure may also be applied to other descriptions and embodiments. That is, all combinations of various elements disclosed in this disclosure fall within the scope of the present invention. Further, the scope of the present invention is not limited by the specific description described below.

To achieve the above objects, an aspect of the present invention provides a cosmetic composition including a bee pollen exosome as an active ingredient.

As used herein, the term “bee pollen” refers to a mixture of pollen, nectar, and salivary secretions when worker bees collect nectar and pollen. Worker bees attach bee pollens to their hind legs and carry the bee pollens to the hive to use as food. The bee pollen contains about 20 types of amino acids, flavonoids, and 16 types of natural vitamins and minerals, etc., and has recently received a lot of attention because it is effective in recovering fatigue, increasing immunity, anti-aging, and antioxidation.

As used herein, the term “exosome” refers to a small vesicle with a membrane structure, which is released from various cells, and is defined as a type of extracellular vesicles (EVs). Exosome contains various biological information such as mRNA and miRNA which are genetic materials, proteins, fats, and metabolites, and contributes to regulating cell function by delivering these materials to target cells. Cells are known to secrete specific messages in the form of exosomes, and to identify them as belonging to specific cells according to surface markers or size of the exosome, and exosomes have higher biocompatibility and in vivo stability than other types of existing particles. Exosomes have been found not only in all biological fluids, including blood, urine, saliva and cell culture of most mammals including humans, but also in plants (microvesicles).

As used herein, the term “bee pollen exosome” is one of plant-derived exosomes. Plant-derived exosomes are natural nanoparticles that contain physiologically active signaling substances secreted by plant cells themselves and help movement between cells and absorption. Exosomes purified from plants are known to be less toxic than mammal-derived exosomes. Specifically, the bee pollen exosome of the present invention may refer to an exosome derived from bee pollen. The above term may be used interchangeably with bee pollen vesicle.

In the present invention, a raw material prepared by adding, to the bee pollen exosome, solvents such as purified water, butylene glycol, 1,2-hexanediol, ethylhexylglycerin, etc., was named a bee pollen exosome raw material.

In the composition of the present invention, the volume (v/v) % of the bee pollen exosome raw material may be 0.001 (v/v) % to 20 (v/v) %, 0.001 (v/v) % to 15 (v/v) %, or 0.001 (v/v) % to 10 (v/v) %, but is not limited thereto.

Further, the bee pollen exosome in the bee pollen exosome raw material of the present invention may be included at a concentration of 1×10⁴ particles/ml to 1×10¹² particles/ml, specifically, 1×10⁵ particles/ml to 1×10¹¹ particles/ml, more specifically, 1×10⁶ particles/ml to 1×10¹⁰ particles/ml, but is not limited thereto.

Further, in the composition of the present invention, the weight (w/w) % of the bee pollen exosome may be 0.0000001% to 1%, 0.0000001% to 0.1%, 0.0000001% to 0.01%, or 0.0000001% to 0.001%, but is not limited thereto.

In the present invention, the cosmetic composition may have effects such as skin whitening, skin elasticity enhancement, wrinkle improvement, skin moisturizing, skin regeneration, skin soothing, skin radiance improvement or anti-inflammation, etc., but is not limited thereto.

Specifically, the cosmetic composition of the present invention may have an increased melanin production-inhibitory effect, as compared to a composition including a pollen extract, a bee pollen extract, or a plant exosome.

The cosmetic composition of the present invention may have an increased NO production-inhibitory effect, as compared to a composition including a pollen extract, a bee pollen extract, or a plant exosome.

The cosmetic composition of the present invention may have an increased cell activation effect, as compared to a composition including a pollen extract, a bee pollen extract, or a plant exosome.

The cosmetic composition of the present invention may have an increased collagen synthesis effect, as compared to a composition including a pollen extract, a bee pollen extract, or a plant exosome.

As used herein, the term “pollen extract” refers to a resulting product such as a liquid component which is obtained by immersing pollens in various solvents and then extracting it at room temperature, a low temperature, or a warm temperature for a predetermined period of time, a solid component obtained by removing the solvents from the liquid component, etc. The pollen is a reproductive organ of vascular plants that produces seeds, and has a structure surrounded by a strong shell to protect the male gametophyte. The pollen is also called flower powder, and is mainly carried by wind or insects to form seeds after fertilization. The pollens include insect-mediated pollens which are mediated by insects and wind-borne pollens which are mediated by wind.

As used herein, the term “bee pollen extract” refers to a resulting product such as a liquid component which is obtained by immersing the bee pollens, which are a mixture of pollen, nectar, and salivary secretions when worker bees collect nectar and pollen, in various solvents and then extracting it at room temperature, a low temperature, or a warm temperature for a predetermined period of time, a solid component obtained by removing the solvents from the liquid component, etc.

As used herein, the term “plant exosome” collectively refers to an exosome or exosome-like extracellular vesicle with a size of about 50 nm to 200 nm, which is extracted from plants. It is known that plant-derived exosomes have lower toxicity and immunogenicity than exosomes secreted from mammalian cells, and have a very low cholesterol content to have very high in vivo stability and biocompatibility. It is also known that plant-derived exosomes are effective in wound healing and skin regeneration due to the antioxidant or protein components derived from plants.

As used herein, the “skin whitening” refers to not only brightening skin tone by inhibiting the synthesis of melanin pigment, but also improving hyperpigmentation of the skin such as spots or freckles caused by ultraviolet rays, hormones, or genetic factors.

Further, the cosmetic composition of the present invention may have an increased melanin production-inhibitory effect, as compared to a composition including a pollen extract, a bee pollen extract, or a plant exosome.

As used herein, the “skin elasticity enhancement” refers to alleviating the loose or saggy skin. Elastic fibers consisting of elastin exist together with collagenous fibers called collagen, and skin elasticity is improved when elastin and collagen sufficiently exist.

The term ‘enhancement’ may mean that the effect on skin elasticity becomes better, or is promoted or improved, but is not limited thereto. In addition, the skin elasticity enhancement of the present invention may mean that the composition of the present invention increases the collagen synthesis effect at least 0.5 times, as compared to a control group without the bee pollen exosome, but is not limited to thereto.

As used herein, the “wrinkle improvement” refers to suppressing or inhibiting the formation of wrinkles on the skin, or relieving wrinkles that have already formed.

Further, the cosmetic composition of the present invention may have an increased collagen synthesis effect, as compared to a composition including a pollen extract, a bee pollen extract, or a plant exosome.

As used herein, the “skin moisturizing” refers to increasing moisture in the skin and keeping the skin moist.

As used herein, the “skin regeneration” refers to regenerating the skin by increasing the expression levels of genes involved in wound healing and genes involved in cell proliferation, which exist in damaged skin areas.

Further, the cosmetic composition of the present invention may have an increased cell activation effect, as compared to a composition including a pollen extract, a bee pollen extract, or a plant exosome.

As used herein, the “skin soothing” refers to giving coolness to the skin, enhancing skin temperature, giving a refreshing feeling to the skin, or giving a cool feeling to the skin, but is not limited thereto.

As used herein, the “skin radiance improvement” refers to making the skin color more radiant and giving the skin a glow. The radiance of skin color may be influenced by a number of external or internal factors. The external factors may include exposure to sunlight, exposure to changes in temperature and humidity, or exposure to contaminants. The internal factors that influence the radiance of skin color may be mentioned as stress, fatigue, hormonal changes, dehydration of the epithelium or damage to the skin barrier function, or even, aging. These external and internal factors tend to darken the skin color, to make the skin color uneven, dull, pale, or even frail, and tend to increase the presence of skin defects or to even worsen skin defects. The cosmetic composition of the present invention may improve skin radiance.

The term ‘improvement’ refers to any action whereby the degree of symptoms is at least reduced or the effect is maximized by the cosmetic composition including the bee pollen exosome of the present invention as an active ingredient. For example, it may mean all that the composition of the present invention inhibits melanin production, inhibits NO production, increases cell activation effect, and increases collagen synthesis effect, as compared to the control group without the bee pollen exosomes, but is not limited to thereto.

As used herein, the “anti-inflammation” refers to the action of suppressing inflammation. The control of the inflammatory response is known to be very complex, and is known to enhance the in vivo repair system and to reduce damage. However, when the inflammatory response continues due to repeated tissue damage or regeneration, ROS and RNS are excessively produced in inflammation-related cells, resulting in permanent genetic changes. As described, ROS and RNS are closely related to the inflammatory response that regulates the actions of various cells in vivo. During the inflammatory process, large amounts of proinflammatory cytokines, nitric oxide (NO), and prostaglandin E2 (PGE2) are generated by inducible nitric oxide synthase (iNOS) and cyclooxygenase-2 (COX-2). Inflammation is a cause of various inflammatory diseases, and the composition including the bee pollen exosome of the present invention may have effects of preventing and improving various inflammatory diseases through anti-inflammatory action.

The cosmetic composition of the present invention may have an increased NO production-inhibitory effect, as compared to a composition including a pollen extract, a bee pollen extract, or a plant exosome.

The cosmetic composition according to the present invention may be prepared into a formulation selected from the group consisting of a solution, an ointment for external use, a cream, a foam, a softening lotion (nutritional emulsion), an astringent lotion, a nourishing lotion, an eye cream, a serum, a nourishing cream, a cleansing cream, a cleansing lotion, a cleansing foam, a cleansing water, a massage cream, a powder, an essence, a pack, a skin adhesive patch, a skin adhesive gel, a powder, an ointment, a suspension, an emulsion, a hair tonic, a hair treatment, a shampoo or rinse, a pack, a softening water, an emulsion, a makeup base, an essence, a soap, a liquid cleanser, a bath preparation, a sunscreen cream, a sun oil, a suspension, an emulsion, a paste, a gel, a lotion, a soap, a surfactant-containing cleanser, an oil, a powder foundation, an emulsion foundation, a wax foundation, and a spray, but is not limited thereto.

In addition, the cosmetic composition of the present invention may further include one or more cosmetically acceptable carriers that are blended in general skin cosmetics, and as common ingredients, for example, oil, water, a surfactant, a moisturizer, low-grade alcohol, a thickener, a chelating agent, a pigment, a preservative, a fragrance, etc. may be appropriately blended, but are not limited thereto.

Cosmetologically acceptable carriers which are included in the cosmetic composition of the present invention vary depending on the formulation.

When the formulation of the present invention is an ointment, a paste, a cream, or a gel, animal oil, plant oil, wax, paraffin, starch, tracant, cellulose derivatives, polyethylene glycol, silicone, bentonite, silica, talc, zinc oxide, or a mixture thereof may be used as the carrier ingredient.

When the formulation of the present invention is a powder or a spray, lactose, talc, silica, aluminum hydroxide, calcium silicate, polyamide powder, or a mixture thereof may be used as carrier ingredient, and specifically, when the formulation is a spray, a propellant such as chlorofluorohydrocarbon, propane/butane, or dimethyl ether may be included.

When the formulation of the present invention is a solution or an emulsion, a solvent, a solubilizing agent, or an emulsifying agent is used as the carrier component, and for example, water, ethanol, isopropanol, ethyl carbonate, ethyl acetate, benzyl alcohol, benzyl benzoate, propylene glycol, 1,3-butylglycol oil may be used, and particularly, cottonseed oil, peanut oil, corn germ oil, olive oil, castor oil and sesame oil, glycerol aliphatic esters, polyethylene glycol, or fatty acid esters of sorbitan may be used.

When the formulation of the present invention is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tracanth, etc. may be used as the carrier ingredient.

When the formulation of the present invention is a soap, alkali metal salts of fatty acids, fatty acid hemiester salts, fatty acid protein hydrolyzate, isethionate, lanolin derivatives, aliphatic alcohols, plant oils, glycerol, sugar, etc. may be used as the carrier ingredient.

The cosmetic composition of the present invention may include auxiliaries commonly used in the cosmetic composition, for example, hydrophilic or lipophilic gelling agents, hydrophilic or lipophilic activators, preservatives, antioxidants, solvents, fragrances, fillers, blockers, pigments, odor-absorbing agents, deodorants, and dyes, etc.

To achieve the above objects, another aspect of the present invention provides a functional food composition including the bee pollen exosome as an active ingredient.

In the present invention, the functional food composition may have effects such as skin whitening, skin elasticity enhancement, wrinkle improvement, skin moisturizing, skin regeneration, skin soothing, skin radiance improvement or anti-inflammation, etc., but is not limited thereto.

The functional food composition may be used in the form of a health functional food, but is not limited thereto.

The composition may include a food supplement acceptable for use in food, in addition to the active ingredient.

As used herein, the “food supplement” refers to a component that may be added auxiliary to food, and may be appropriately selected and used by a person skilled in the art as it is added to prepare each formulation of health functional food. Examples of the food supplement may include various nutrients, vitamins, minerals (electrolytes), flavoring agents such as synthetic and natural flavors, etc., colorants and fillers, pectic acid and salts thereof, alginic acid and salts thereof, organic acids, and protective colloidal thickeners, pH adjusters, stabilizers, preservatives, glycerin, alcohol, carbonating agents used in carbonated drinks, etc., but the type of the food supplement of the present invention is not limited to the above examples.

The functional food composition of the present invention may include a health functional food. As used herein, the “health functional food” refers to foods which are prepared and processed in the form of tablets, capsules, powder, granules, liquids, pills, etc. by using raw materials or ingredients having useful functionality in the human body. The “functionality” as used herein refers to controlling nutrients for the structure and functions of the human body or providing useful effects of hygienic purposes, such as physiological actions, etc. The health functional food of the present invention may be prepared by a method commonly used in the art and may be prepared by adding raw materials and ingredients that are commonly added in the art. In addition, the health functional food may be prepared into any formulation without limitation as long as it is acceptable as a health functional food. The food composition of the present invention may be prepared into various dosage forms, and unlike general pharmaceuticals, the health functional food uses foods as raw materials, and thus has advantages of not causing side effects that may occur during long-term administration of a drug, and of having excellent portability. Accordingly, the health functional food of the present invention may be ingested as a supplement for skin whitening, skin elasticity enhancement, wrinkle improvement, skin moisturizing, skin regeneration, skin soothing, skin radiance improvement or anti-inflammation.

There is no limitation on the types of health functional food of the present invention, and the health functional food may include all foods in the ordinary acceptation, and may be used interchangeably with terms known in the art, such as functional food. In addition, the health functional food of the present invention may be prepared by mixing with known additives and other appropriate auxiliary ingredients that may be included in the food at the option of a person skilled in the art. Examples of the food that may be added include meats, sausages, bread, chocolate, candy, snacks, confectionery, pizza, ramen, other noodles, gum, dairy products including ice cream, various soups, beverages, tea, drinks, and alcoholic beverages, and vitamin complexes, etc. The food also includes foods used as feeds for animals.

To achieve the above objects, still another aspect provides a quasi-drug composition including the bee pollen exosome as an active ingredient.

In the present invention, the quasi-drug composition may have the effects of skin whitening, skin elasticity enhancement, wrinkle improvement, skin moisturizing, skin regeneration, skin soothing, skin radiance improvement or anti-inflammation, etc., but is not limited thereto.

In addition to the above ingredients, the quasi-drug composition of the present invention may further include pharmaceutically acceptable carriers, excipients, or diluents, as needed. The pharmaceutically acceptable carriers, excipients, or diluents are not limited as long as they do not impair the effect of the present invention, and may include, for example, fillers, extenders, binders, wetting agents, disintegrants, surfactants, lubricants, sweeteners, fragrances, preservatives, etc.

Representative examples of the pharmaceutically acceptable carriers, excipients, or diluents of the present invention may include lactose, dextrose, sucrose, sorbitol, mannitol, xylitol, maltitol, starch, gelatin, glycerin, acacia rubber, alginate, calcium phosphate, calcium carbonate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, propylene glycol, polyethylene glycol, plant oils, injectable esters, Witepsol, Macrogol, Tween 61, cocoa butter, laurin butter, etc.

In addition, when the composition including the bee pollen exosome of the present invention as an active ingredient is used as a quasi-drug, it may further include one or more active ingredients exhibiting the same or similar functions. For example, the composition may include known ingredients for skin whitening, elasticity enhancement, wrinkle improvement, skin regeneration, moisturizing, skin soothing, skin radiance improvement and anti-inflammation. When additional ingredients for skin whitening, elasticity enhancement, wrinkle improvement, skin regeneration, moisturizing, skin soothing, skin radiance improvement and anti-inflammation are included, the composition of the present invention may have the further improved effects of skin whitening, skin elasticity enhancement, wrinkle improvement, skin regeneration, moisturizing, skin soothing, skin radiance improvement and anti-inflammation. When adding the above ingredients, skin safety due to combined use, ease of formulation, and stability of the active ingredients may be taken into consideration. The quasi-drug composition may include one ingredient or two or more ingredients selected from the group consisting of substances inhibiting tyrosinase enzyme activity, such as Kojic acid, arbutin, etc., hydroquinone, and vitamin-C (L-ascorbic acid), which are whitening ingredients known in the art; retinoic acid, TGF, animal placenta-derived proteins, betulinic acid, and a chlorella extract which are skin elasticity, wrinkle improving or moisturizing ingredients known in the art; and derivatives thereof and various plant extracts. The additional ingredients may be included in an amount of 0.0001% by weight to 10% by weight, based on the total weight of the composition, and the content range may be adjusted according to requirements such as skin safety, ease of use, etc.

The quasi-drug composition of the present invention may be exemplified by disinfecting cleaners, shower foams, ointments, wet wipes, coating agents, etc., but is not limited thereto, and the formulation method, dosage, method of use, components, etc. of the quasi-drug may be appropriately selected from common techniques known in the art.

In addition, the quasi-drug composition including the bee pollen exosome of the present invention as an active ingredient may be used in a method for skin whitening, skin elasticity improvement or wrinkle improvement, or skin moisturizing, the method including the step of applying the composition to a subject's skin. The subject includes mammals, including rats, livestock, humans, etc., without limitation.

To achieve the above objects, still another aspect provides a method of preparing the bee pollen exosome, the method including the step of extracting bee pollens at a low temperature; and isolating the exosome from the extract.

Specifically, in the step of extracting the bee pollen at a low temperature, the low temperature may be 4° C. to 25° C., 4° C. to 20° C., or 4° C. to 15° C., but is not limited thereto. In addition, in the above step, in order to minimize destruction of the active ingredients of the bee pollen before the step of low-temperature extracting, it may be ground using a millstone method, but is not limited thereto as long as it is a grinding method commonly used.

Additionally, in the step of isolating the exosome from the extract, precipitates may be removed using centrifugation. Thereafter, the exosome may be purified using a filtration device, but any suitable separation or purification method known in the art may be performed without limitation.

To achieve the above objects, still another aspect provides a method of improving the skin, the method including the step of applying, to a subject's skin, the composition including the bee pollen exosome of the present invention as an active ingredient.

The term ‘bee pollen exosome’ is described as above. According to the skin improvement method, the composition including the bee pollen exosome of the present invention is confirmed to have the effects of i) inhibiting melanin production, ii) inhibiting NO production, iii) increasing cell activity; and/or iv) increasing collagen synthesis, as compared to a composition including a pollen extract, a bee pollen extract, or a plant exosome, thereby providing a method for skin whitening, skin elasticity enhancement, wrinkle improvement, skin moisturizing, skin regeneration, skin soothing, skin radiance improvement or anti-inflammation, but is not limited thereto.

To achieve the above objects, still another aspect provides a bee pollen exosome which is prepared by the method.

To achieve the above objects, still another aspect provides a composition including the bee pollen exosome.

To achieve the above objects, still another aspect provides use of the bee pollen exosome or the composition including the same in improving the skin.

The term ‘bee pollen exosome’ is described as above. The use of the present invention in improving the skin may refer to the use for skin whitening, skin elasticity enhancement, wrinkle improvement, skin moisturizing, skin regeneration, skin soothing, skin radiance improvement or anti-inflammation, but is not limited thereto.

### Advantageous Effects

A cosmetic composition including a bee pollen exosome of the present invention as an active ingredient has excellent effects of skin whitening, skin elasticity enhancement, wrinkle improvement, skin moisturizing, skin regeneration, skin soothing, skin radiance improvement or anti-inflammation. In particular, the composition including the bee pollen exosome of the present invention has excellent melanin production inhibitory effects, NO production inhibitory effects, cell activation effects, and collagen synthesis effects, as compared to a composition including a pollen extract, a bee pollen extract, or a plant exosome, and thus it is highly applicable as a cosmetic composition for whitening the skin, enhancing elasticity or improving wrinkles, moisturizing the skin, regenerating the skin, soothing the skin, improving skin radiance, and anti-inflammation.

### BRIEF DESCRIPTION OF THE DRAWING

FIG. 1 shows a flow chart of a process of preparing a bee pollen exosome raw material;

FIG. 2 shows a graph showing the MMP-1 inhibitory effect of a bee pollen exosome;

FIG. 3 shows the evaluation of elasticity improvement of a serum containing 5% bee pollen exosomes;

FIG. 4 shows the degree of improvement of wrinkles of under-eyes, nasolabial folds, chin, or neck by the serum containing 5% bee pollen exosomes;

FIG. 5 shows the results of a multi-faceted radiance test of the serum formulation containing 5% bee pollen exosomes;

FIG. 6 shows the results of a multi-faceted radiance test when the serum containing 5% bee pollen exosomes+a cream containing 3% bee pollen exosomes were used in combination;

FIG. 7 shows the results of a multi-faceted radiance test of the serum containing 5% bee pollen exosomes vs combination use of the serum containing 5% bee pollen exosomes & the cream containing 3% bee pollen exosomes; and

FIG. 8 shows changes in the multi-faceted radiance test of the serum containing 5% bee pollen exosomes vs combination use of the serum containing 5% bee pollen exosomes & the cream containing 3% bee pollen exosomes.

### DETAILED DESCRIPTION OF PREFERRED EMBODIMENTS

Hereinafter, the present invention will be described in more detail with reference to the following exemplary embodiments. However, the following exemplary embodiments are only for illustrating the present invention, and the scope of the present invention is not intended to be limited by the following exemplary embodiments.

### Preparation Example 1: Preparation of Bee Pollen Exosome

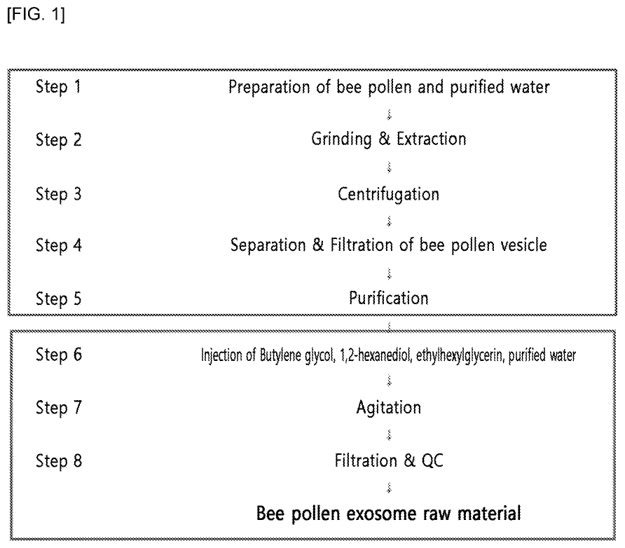

To prepare a bee pollen exosome of the present invention, bee pollens collected by bees during the flowering season (around March to May) in Seorak Mountain were used. In detail, in order to minimize the destruction of the active ingredients, the bee pollens were ground using a traditional millstone method to destroy the cell walls, and then extracted with purified water at a low temperature for 24 hours to 72 hours. The low temperature may be 4° C. to 25° C., specifically, 4° C. to 15° C. Thereafter, the precipitate was removed by centrifugation at 8,000 rpm or 5,000, and the supernatant was taken, and then filtered using a filter having a pore size of 0.2 μm to 5.0 μm to obtain a filtrate containing exosomes. Purified exosomes were separated by molecular weight cut-off using a tangential flow filtration device (TFF device) with a 100 kDa filter and washing (Step 1 to Step 5 in FIG. 1).

### Preparation Example 2: Preparation of Bee Pollen Exosome Raw Material

A raw material prepared by adding, to 0.00003% of the bee pollen exosome obtained from Preparation Example 1, 10% of butylene glycol, 2% of 1,2-hexanediol, 0.05% of ethylhexylglycerin, and purified water (to 100%), based on wt %, was named ‘bee pollen exosome raw material’ (Steps 6 to 8 in FIG. 1). The density of the bee pollen exosome raw material may be 1.004 g/ml.

### Example 1: Verification of Melanin Production Inhibitory Effect

In order to verify the whitening effect of the bee pollen exosome raw material prepared in Preparation Example 2, the melanin production-inhibitory effect was measured as follows.

In detail, the whitening efficacy of the bee pollen exosome raw material was evaluated by measuring the degree of reduction in the amount of melanin pigment present in melanocytes. The bee pollen exosome raw material was diluted to 0.01% and 0.1% concentrations (v/v %), and B16f10 melanocytes were treated therewith, and cultured for 72 hours, and melanin was quantified to evaluate the whitening efficacy. The melanin content was calculated based on the total protein amount, and the melanin production inhibition rate was calculated as %, based on a DMSO control group. 200 ppm of arbutin was treated as a positive control group.

Further, it was intended to determine whether the melanin production inhibitory effect of the bee pollen exosome raw material was superior to those of a bee pollen extract raw material, a pollen extract raw material, and a plant-derived exosome raw material, which are control groups. In detail, the bee pollen extract raw material was prepared by grinding the same amount of bee pollens in the same manner as in preparing the bee pollen exosome, and by extracting using a general low-temperature extraction method of using purified water for 24 hours to 72 hours under the same temperature conditions (4° C. to 25° C.). Without the centrifugation step required to obtain exosomes, solvents (10% butylene glycol, 2% 1,2-hexanediol, 0.05% ethylhexylglycerin, and purified water (to 100%), based on wt %) were immediately added to obtain the same composition as the bee pollen exosome raw material, thereby preparing the raw material. The pollen extract raw material was prepared by performing the low-temperature extraction of commercially purchased pollens (dried wild rose pollen product) under the same conditions (grinding, extraction with purified water at 4° C. to 25° C. for 24 hours to 72 hours), and then adding the solvents of the same composition (10% butylene glycol, 2% 1,2-hexanediol, 0.05% ethylhexylglycerin, and purified water (to 100%), based on wt %). Further, the plant exosome raw material was prepared by extracting exosomes using another plant (carrot) instead of the bee pollens in the same technique (Proceed to Step 5 in FIG. 1 using carrots instead of the bee pollens), and then adding the same solvents (10% butylene glycol, 2% 1,2-hexanediol, 0.05% ethylhexylglycerin, and purified water (to 100%), based on wt %).

TABLE 1

| Concen- | Bee pollen | Bee pollen | Pollen | Plant exosome |
|---|---|---|---|---|
| tration | exosome raw | extract raw | extract raw | (carrot) raw |
| (v/v %) | material | material | material | material |
| 0.01% | 37.6 | 25.2 | 18.0 | 10.4 |
| 0.1% | 38.0 | 28.0 | 19.9 | 16.5 |

As a result, as shown in Table 1, it was confirmed that the group treated with the bee pollen exosome raw material showed the highest melanin production inhibition rate (%). The positive control group (200 ppm of arbutin) showed 38.06% inhibition of melanin synthesis.

### Example 2: Verification of Skin Soothing and Anti-Inflammatory Effects

In order to verify the skin soothing and anti-inflammatory effects of the bee pollen exosome raw material prepared in Preparation Example 2, the NO production inhibitory effect was measured as follows, and compared with those of the pollen extract raw material, the bee pollen extract raw material, and the plant-derived exosome raw material which are the control groups.

In detail, the NO production inhibitory effects of the above samples were measured using Raw 264.7 cells. As a positive control, 20 mg/ml L-NMMA1 (NOS inhibitor) was used, and the samples were diluted to 0.01% and 0.1% concentrations (v/v %) and treated for 30 minutes, followed by treatment with 1 μg/ml LPS and culturing for 24 hours. Then, the ability to inhibit NO production was evaluated using a NO quantification kit as follows. All results were expressed as mean±standard deviation (SD) after three or more independent experiments.

NO production inhibitory ability (%)=(1−(slope of NO production amount with sample addition))/(NO production amount without sample addition)×100

TABLE 2

| Concen- | Bee pollen | Bee pollen | Pollen | Plant exosome |
|---|---|---|---|---|
| tration | exosome raw | extract raw | extract raw | (carrot) raw |
| (v/v %) | material | material | material | material |
| 0.01% | 48.32 | 38.32 | 34.38 | 17.47 |
| 0.1% | 57.20 | 44.24 | 38.18 | 14.59 |

As a result, as shown in Table 2, it was confirmed that the group treated with the bee pollen exosome raw material showed the highest NO production inhibition rate (%). The positive control group (200 ppm L-NMMA1) showed 59.17% inhibition ability. These results indicate that the group treated with the bee pollen exosome raw material showed the most excellent skin soothing and anti-inflammatory effects.

### Example 3: Verification of Cell Activation Effect

To verify the cell proliferation effect of the bee pollen exosome raw material prepared in Preparation Example 2, the cell activation effect was measured as follows.

In detail, human dermal fibroblast (HDF) cells were counted at 1×10⁴ cells/well using a hemacytometer, and then dispensed in a 96 well plate. The pollen exosome raw material was added to each well of HDF cells which were cultured for 24 hours, and cultured in 100 ul of serum free DMEM medium at 37° C. in a 5% CO₂ incubator for 24 hours. 10 ul of CCK-8 reagent was added to each well, and after incubation for 1 hour, absorbance was measured at 450 nm. The increased cell activity was expressed as a percentage (%) based on the untreated control group. 5% FBS was used as a positive control.

Further, it was intended to determine whether the cell activation effect of the bee pollen exosome raw material was superior to those of the control groups, the pollen extract raw material, the bee pollen extract raw material, and the plant-derived exosome raw material. All of the above samples were diluted to 0.01% and 0.1% concentrations (v/v %) and then evaluated.

TABLE 3

| Concen- | Bee pollen | Bee pollen | Pollen | Plant exosome |
|---|---|---|---|---|
| tration | exosome raw | extract raw | extract raw | (carrot) raw |
| (v/v %) | material | material | material | material |
| 0.01% | 23.30 | 12.20 | 2.05 | 2.34 |
| 0.1% | 29.20 | 15.42 | 2.50 | 7.20 |

As a result, as shown in Table 3, it was confirmed that when treated with the bee pollen exosome raw material, the cell activation effect was superior to those of treatment with other extracts. When 5% FBS was treated as the positive control group, the cell activity was increased by 34.77%.

### Example 4: Evaluation of Collagen Synthesis Efficacy

To verify the collagen synthesis effect of the bee pollen exosome raw material prepared in Preparation Example 2, measurements were performed as follows.

In detail, the collagen synthesis effect was evaluated using human dermal fibroblast (HDF) cells. After pre-treating the cells with the bee pollen exosome raw material sample for 48 hours, collagen synthesis efficacy was measured using a human procollagen type I C-peptide (PIP) EIA kit. The collagen synthesis efficacy of the sample was determined by calculating the increased value in percentage (%), as compared to the untreated control group, and the results are shown in a table. 10 ng/ml TGF-b was used as a positive control group.

In addition, it was intended to confirm whether the collagen synthesis effect of the bee pollen exosome raw material was superior to those of the pollen extract raw material, the bee pollen extract raw material, and the plant-derived exosome raw material, which were control groups. All of the above samples were diluted to 0.01% and 0.1% concentrations (v/v %) and evaluated.

TABLE 4

| Concen- | Bee pollen | Bee pollen | Pollen | Plant exosome |
|---|---|---|---|---|
| tration | exosome raw | extract raw | extract raw | (carrot) raw |
| (v/v %) | material | material | material | material |
| 0.01% | 13.30 | 8.40 | 2.14 | −1.20 |
| 0.1% | 18.20 | 8.92 | 1.21 | 1.15 |

As a result, as shown in Table 4, it was confirmed that when treated with the bee pollen exosome raw material, the collagen synthesis effect was superior to those of treatment with other extracts. When the positive control TGF-b was treated, the collagen synthesis was increased by 29.87%, as compared to the untreated group.

### Example 5: Evaluation of Anti-Aging Efficacy (Verification of MMP-1 Inhibitory Efficacy)

To verify the anti-aging effect of the bee pollen exosome raw material prepared in Preparation Example 2, the MMP-1 inhibitory efficacy was evaluated as follows.

In detail, the inhibitory effect of MMP-1, which increases upon UV irradiation, was evaluated using human dermal fibroblast (HDF) cells. HDF cells were seeded in a 6-well plate, and 24 hours later, irradiated with UVB at 25 mJ/cm², and treated with the pollen exosome raw material sample, and cultured for 24 hours to measure the MMP-1 expression level using an MMP-1 DuoSet ELISA kit. The MMP-1 efficacy of the bee pollen exosome raw material sample was calculated, compared to the untreated control group, and the value was measured, and the results were graphed. 0.3 ppm retinoic acid (RA) was used as a positive control group.

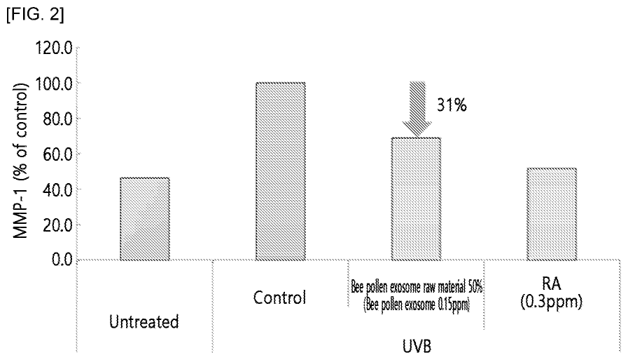

As a result, as shown in FIG. 2, the MMP-1 expression level decreased by 31% in the group treated with the bee pollen exosome raw material, as compared to the untreated control group, indicating excellent MMP-1 inhibitory efficacy.

### Preparation Example 3: Preparation of Bee Pollen Exosome-Containing Serum and Cream

A serum and a cream, each containing the bee pollen exosome raw material, were prepared with the composition shown in Tables 5 and 6 below.

TABLE 5
Serum containing bee pollen exosome

| | | | | | Comparative |
|---|---|---|---|---|---|
| | Example | | | | Example |
| Section (wt %) | 1 | 2 | 3 | 4 | 1 |
| Bee pollen | 1 | 3 | 5 | 10 | 0 |
| exosome raw | | | | | |
| material | | | | | |
| Butylene glycol | 5 | 5 | 5 | 5 | 5 |
| Glycerin | 10 | 10 | 10 | 10 | 10 |
| Propanediol | 6 | 6 | 6 | 6 | 6 |
| Sodium polyacrylate | 0.02 | 0.02 | 0.02 | 0.02 | 0.02 |
| Squalane | 3.5 | 3.5 | 3.5 | 3.5 | 3.5 |
| Fragrance | 0.24 | 0.24 | 0.24 | 0.24 | 0.24 |
| Purified water | re- | re- | re- | re- | re- |
| | mainder | mainder | mainder | mainder | mainder |

TABLE 6
Cream containing bee pollen exosome

| | | | | Comparative |
|---|---|---|---|---|
| | Example | | | Example |
| Section (wt %) | 5 | 6 | 7 | 2 |
| Bee pollen exosome | 1 | 2 | 3 | 0 |
| raw material | | | | |
| Dipropylene glycol | 5 | 5 | 5 | 5 |
| Glycerin | 3 | 3 | 3 | 3 |
| Carbomer | 0.2 | 0.2 | 0.2 | 0.2 |
| Tromethamine | 0.2 | 0.2 | 0.2 | 0.2 |
| Glyceryl stearate | 2 | 2 | 2 | 2 |
| Caprylic/Capric triglyceride | 4 | 4 | 4 | 4 |
| Fragrance | 0.2 | 0.2 | 0.2 | 0.2 |
| Purified water | re- | re- | re- | re- |
| | mainder | mainder | mainder | mainder |

### Example 6: Evaluation of Improvement in Elastic Ring of Serum Containing 5% Bee Pollen Exosome Raw Material

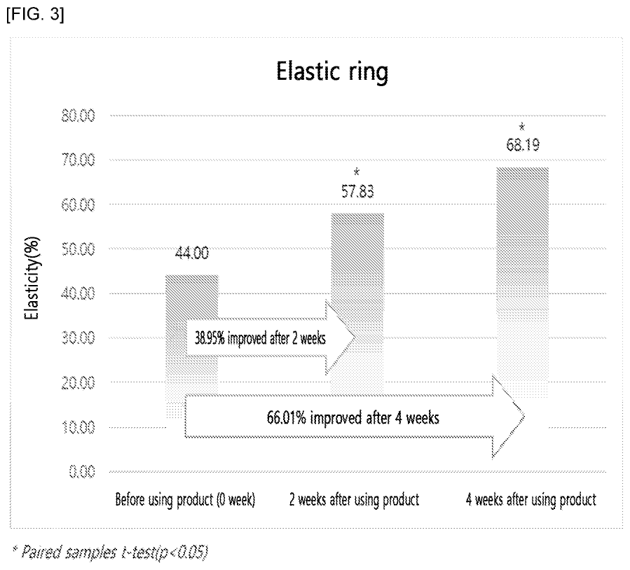

Cutiscan CS 100 was used to measure skin elasticity in the same area of the facial area. The skin was uniformly suctioned in all directions with a constant negative pressure provided inside the device for a few seconds, and then the applied pressure was released again for several seconds. During the suction and recovery time, a camera with lights inside a probe monitors the displacement of each pixel by an optical flow algorithm, and the unit of measurement is Pixel. The V1, V2, and V3 values were calculated, and the average values of 0° (360°), 45°, 90°, 135°, 180°, 228°, 270°, 315°, 360° (0°) were used. V1 evaluates the firmness of the skin when suctioned, V2 evaluates the ability of the skin to retract when released, and V3 represents a curve of the ratio of V2/V1 expressed as %. The parameters representing elasticity are the V2 value and the V3 value, and the V3 value representing elasticity is used by using the V1 value and V2 value. The results of elasticity improvement are shown in a graph of FIG. 3 using the V3 value (unit %) representing elasticity as the Y axis. The 3D graph image has a cylindrical ‘ring’ shape that visualizes suction and recovery from 0° to 360°, and thus the test was conducted considering it as an ‘elastic ring’ in the corresponding test. In the test, when the V3 value increases before using the test product (0 week), 2 weeks after using the product, and 4 weeks after using the product, it means that the elasticity is improved.

As a result, as shown in FIG. 3, it was confirmed that when the serum containing 5% bee pollen exosome raw material was used, 38.95% improvement was observed 2 weeks after using the product, and 66.01% improvement was observed 4 weeks after using the product.

### Example 7: Verification of Improvement of Wrinkles of Under-Eyes, Nasolabial Folds, Chin, and Neck by Serum Containing 5% Bee Pollen Exosome Raw Material

To verify the wrinkle improvement effect of the serum and cream containing bee pollen exosome raw material prepared in Preparation Example 2, the effects of improving wrinkles of under-eyes, nasolabial folds, chin, and neck were evaluated.

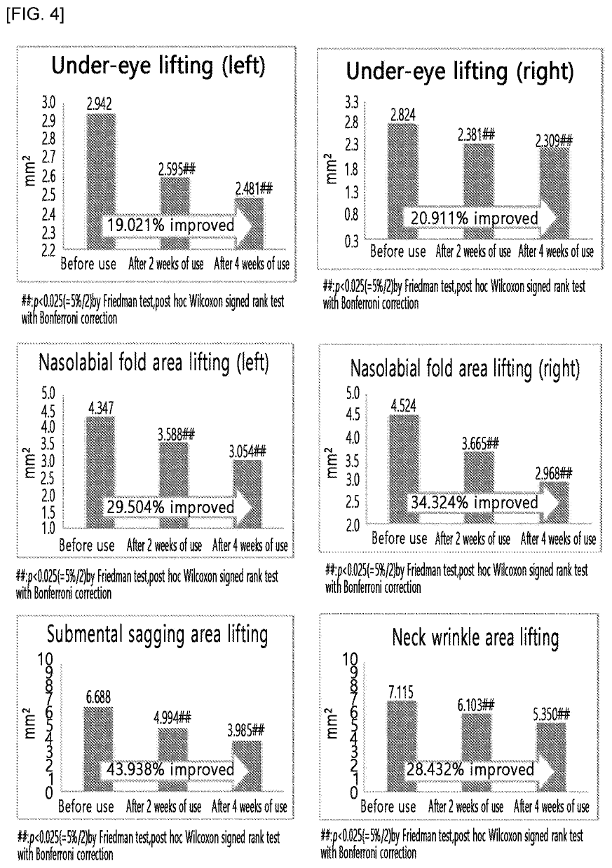

As a result, as shown in FIG. 4, the left and right under-eye lifting, left and right nasolabial fold area lifting, submental sagging area lifting, and neck wrinkle area lifting were performed by photographing subjects' left and right under-eyes, left and right nasolabial folds, double chin, and neck using Antera 3D CS (Miravex Ltd., Ireland) which is a 3D skin imaging device before and after using the test product, and the captured images were analyzed in a Depressions-Medium mode. The parameter is the volume (mm³) value, and as the analysis value increases, it means that the product is effective in lifting the left and right under-eye areas, lifting the left and right nasolabial folds, lifting the sagging areas under the chin, and lifting the neck wrinkles.

As a result of the evaluation of under-eye lifting (left, right), there was a statistically significant improvement after 2 weeks and 4 weeks of use, as compared to before use, and it was confirmed that the improvement rate was 16.820% (left) and 14.029% (right) after 2 weeks of use, and the improvement rate was 19.021% (left) and 20.911% (right) after 4 weeks of use, as compared to before use (0 week) of the product.

As a result of the evaluation of nasolabial fold area lifting (left, right), there was a statistically significant improvement after 2 weeks and 4 weeks of use, as compared to before use, and it was confirmed that the improvement rate was 17.490% (left) and 22.121% (right) after 2 weeks of use, and the improvement rate was 29.504% (left) and 34.324% (right) after 4 weeks of use, as compared to before use (0 week) of the product.

As a result of the evaluation of the chin sagging area lifting, there was a statistically significant improvement after 2 weeks and 4 weeks of use, as compared to before use, and it was confirmed that the improvement rate was 24.617% after 2 weeks of use, and the improvement rate was 43.938% after 4 weeks of use, as compared to before use (0 week) of the product.

As a result of the evaluation of the neck wrinkle area lifting, there was a statistically significant improvement after 2 weeks and 4 weeks of use, as compared to before use, and it was confirmed that the improvement rate was 14.364% after 2 weeks of use, and the improvement rate was 28.432% after 4 weeks of use, as compared to before use (0 week) of the product.

### Example 8: Multi-Faceted Radiance Evaluation of Serum and Cream Containing Bee Pollen Exosome

The gloss (G.U) of the test area (facial area) was measured using a Skin-Glossymeter GL200WL. Skin-Glossymeter GL200WL measures the gloss value by reflection of light sent to the skin surface. In the evaluation, the changes in radiance before and immediately after using the product were compared and analyzed, and as the measurement value increases, it means that the radiance of the skin is improved. The unit of measurement is diffuse scattering correction (DSC, G.U). In the multi-faceted radiance evaluation, the changes before and immediately after using the product were compared and analyzed.

Images were taken for the facial area using Mark-Vu (PSI Plus). The same site on the front of the face was photographed before and immediately after using the product, and SL was used.

### 8-1. Multi-Faceted Radiance Evaluation of Serum Formulation Containing 5% Bee Pollen Exosome Raw Material

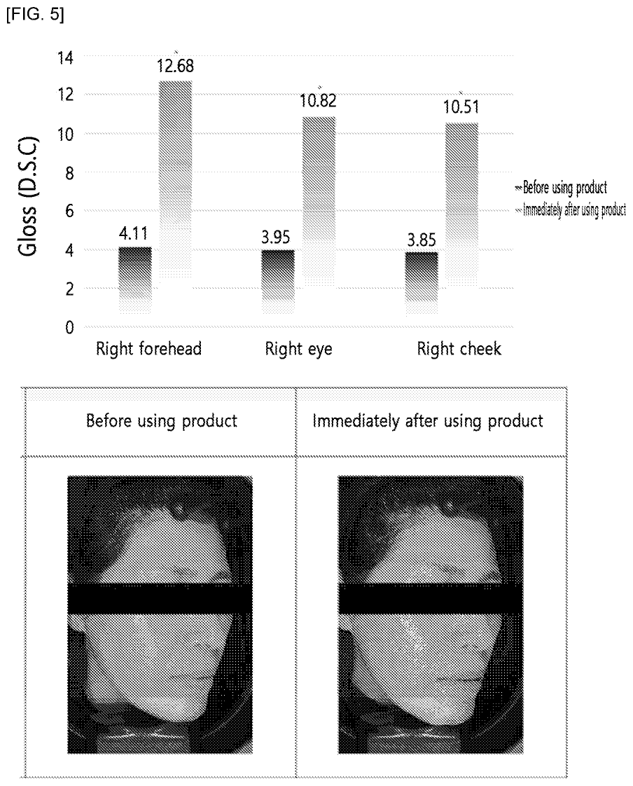

As a result, as shown in FIG. 5, as a result of using the serum containing 5% bee pollen exosome raw material, improvement rates of 226.67% in the right forehead, 184.91% in the right eye area, and 187.69% in the right cheek were observed immediately after using the product, as compared to before using the product.

### 8-2. Multi-Faceted Radiance Evaluation of Combination Use of Serum Containing 5% Bee Pollen Exosome Raw Material & Cream Containing 3% Bee Pollen Exosome Raw Material

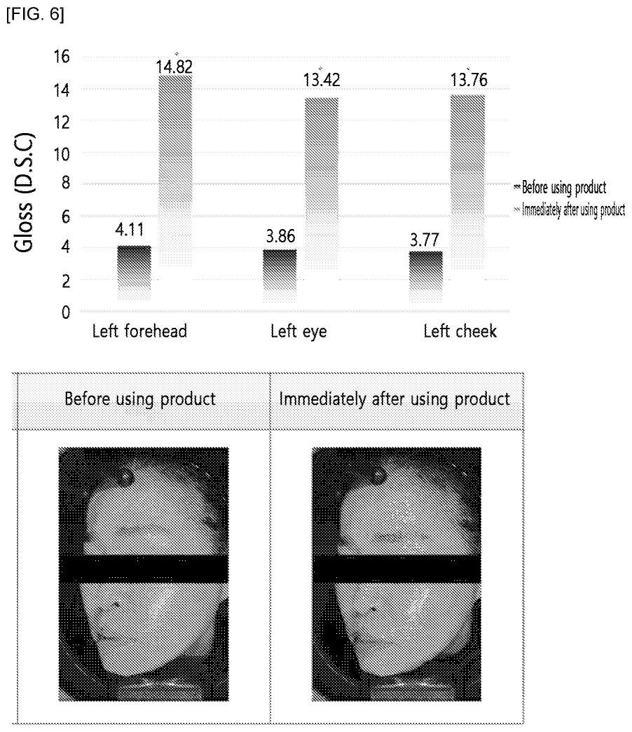

As a result, as shown in FIG. 6, as a result of using the serum containing 5% bee pollen exosome raw material and the cream containing 3% bee pollen exosome raw material in combination, improvement rates of 273.95% in the right forehead, 259.87% in the left eye area, and 283.22% in the left cheek were observed immediately after using the product, as compared to before using the product.

### 8-3. Multi-Faceted Radiance Evaluation of Serum Containing 5% Bee Pollen Exosome Raw Material VS Combination Use of Serum Containing 5% Bee Pollen Exosome Raw Material & Cream Containing 3% Bee Pollen Exosome Raw Material

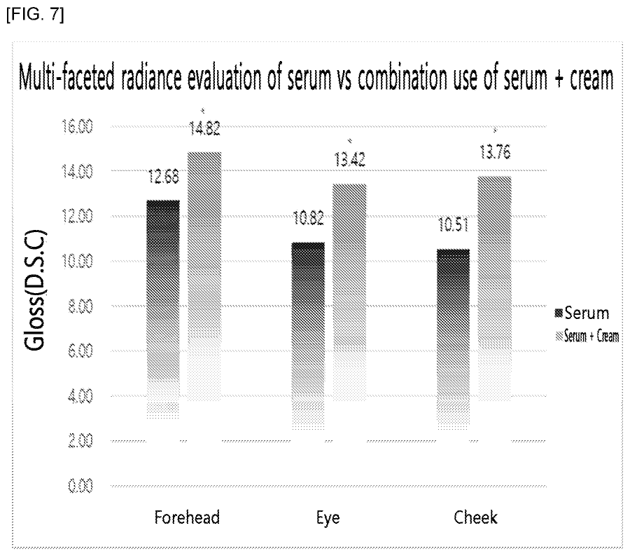

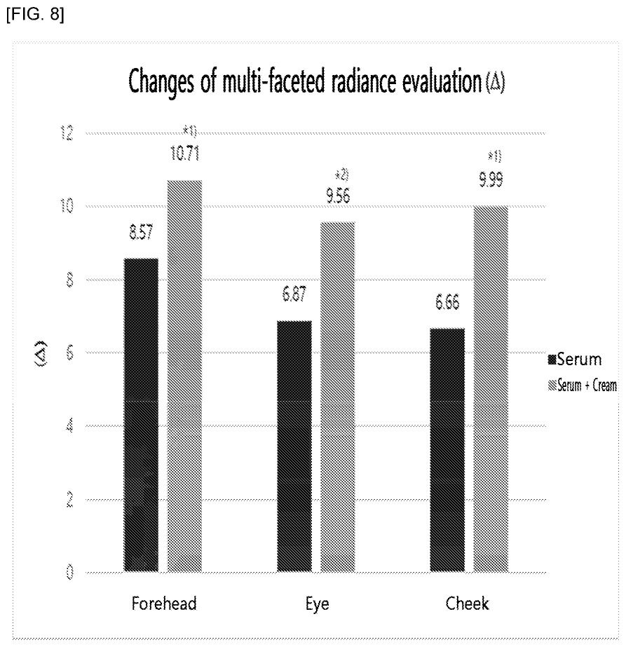

As a result, as shown in FIG. 7, as a result of using the serum containing 5% bee pollen exosome raw material and using the serum containing 5% bee pollen exosome raw material+the cream containing 3% bee pollen exosome raw material in combination, there was a statistically significant level of radiance improvement both in the single use and the combination use. As shown in FIG. 8, the change in each multi-faceted radiance evaluation was high when the serum containing 5% bee pollen exosome raw material & the cream containing 3% bee pollen exosome raw material were used in combination, as compared to single use of the serum containing 5% of bee pollen exosome raw material.

Based on the above description, it will be understood by those skilled in the art that the present invention may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. A composition comprising a bee pollen exosome as an active ingredient.

2. The composition of claim 1, wherein the composition is for skin whitening, skin elasticity enhancement, wrinkle improvement, skin moisturizing, skin regeneration, skin soothing, skin radiance improvement or anti-inflammation.

3. The composition of claim 1, wherein the bee pollen exosome is included in an amount of 0.0000001% by weight to 1% by weight, based on the total weight of the composition.

4. The composition of claim 1, wherein the composition has one or more effects selected from the following i) to iv), as compared to a composition including a pollen extract, a bee pollen extract, or a plant exosome:
i) inhibiting melanin production,
ii) inhibiting NO production,
iii) increasing cell activity; and/or
iv) increasing collagen synthesis.

5. The composition of claim 1, wherein the composition is for a cosmetic, a functional food or a quasi-drug.

6. (canceled)

7. (canceled)

8. A method of preparing a bee pollen exosome, the method comprising the steps of:
extracting a bee pollen at a low temperature; and
isolating the exosome from the extract.

9. A bee pollen exosome prepared by the preparation method of claim 8.

10. A composition comprising the bee pollen exosome of claim 9.

11. A method for producing a cosmetic composition comprising a bee pollen exosome as an active ingredient.

12. The method of claim 11, wherein the composition has one or more effects selected from the following i) to iv), as compared to a composition including a pollen extract, a bee pollen extract, or a plant exosome:
i) inhibiting melanin production,
ii) inhibiting NO production,
iii) increasing cell activity; and/or
iv) increasing collagen synthesis.

13. A skin improvement method comprising applying a composition of claim 1 to the skin of a subject.

14. The method of claim 13, wherein the skin improvement is for skin whitening, skin elasticity enhancement, wrinkle improvement, skin moisturizing, skin regeneration, skin soothing, skin radiance improvement or anti-inflammation.

15. The method of claim 13, wherein the skin improvement has one or more effects selected from the following i) to iv):
i) inhibiting melanin production,
ii) inhibiting NO production,
iii) increasing cell activity; and/or
iv) increasing collagen synthesis.
